(19) United States
(12) Patent Application Publication
(10) Pub. No.: US 2021/0299468 A1
(43) Pub. Date: Sep. 30, 2021

(54) BRACHYTHERAPY SYSTEM

(71) Applicant: Carl Zeiss Meditec AG, Jena (DE)

(72) Inventors: Michael Symalla, Aalen (DE); Norman Roeder, Doebritschen (DE)

(21) Appl. No.: 17/214,604

(22) Filed: Mar. 26, 2021

(30) Foreign Application Priority Data
Mar. 26, 2020 (DE) 10 2020 108 352.1

(51) Int. Cl. A61N 5/10; G01T 1/161

(52) U.S. Cl. CPC A61N 5/1001; G01T 1/161

(57) ABSTRACT

The disclosure relates to a brachytherapy system. The brachytherapy system comprises a radiation generation apparatus for generating x-ray radiation, a controller for controlling the radiation generation apparatus, an applicator, which is embodied in such a way that it can be placed on the radiation generation apparatus, and an information transmission system, which is configured to capture an information item embodied in or at the applicator if the applicator has been placed on the radiation generation apparatus and to transmit the captured information item to the controller. The controller is configured to control the generation of x-ray radiation and the output of warnings on the basis of the received information item.

### CROSS REFERENCE TO RELATED APPLICATION

This application claims priority of German patent application no. 10 2020 108 352.1, filed Mar. 26, 2020, the entire content of which is incorporated herein by reference.

### TECHNICAL FIELD

The present disclosure lies in the field of medical radiotherapy and relates to a brachytherapy system.

### BACKGROUND

Brachytherapy is a method for therapeutic treatment of tissue in the interior of a human or animal body by means of x-ray radiation. Brachytherapy is performed, in particular, following a resection of tumour tissue. Brachytherapy is performed using a brachytherapy system.

Conventional brachytherapy systems include a radiation generation apparatus and an applicator.

The radiation generation apparatus serves to generate x-ray radiation. A conventional radiation generation apparatus includes a particle beam system, which is able to generate a high energy particle beam. The particle beam is directed through a tube of the radiation generation apparatus with a length of a few centimetres at an x-ray material, which is arranged at the end of the tube. As a result of the interaction of the particle beam with the x-ray material, the latter generates x-ray radiation, which is provided for the therapeutic treatment of tissue.

So that the x-ray radiation generated by the x-ray material of the tip of the tube can be applied in the interior of a body, the tube is inserted into the body. To this end, the tube is surrounded by an applicator that is able to be placed on the radiation generation apparatus, the applicator firstly representing a sterile barrier and secondly protecting the tube of the radiotherapy device.

Conventionally, an applicator is selected by a person and placed on the radiation generation apparatus prior to the irradiation. As a rule, the selection has to be implemented according to a radiation treatment plan, which is set by a physician and which specifies the applicator to be used for the irradiation. By way of example, the radiation treatment plan specifies the size and type of applicator to be used for the irradiation and the irradiation duration and irradiance.

A problem of this conventional method is that the person can select an applicator that does not correspond to the radiation treatment plan and use the applicator for the irradiation. This can lead to a flawed therapy. Moreover, there is no reliable option for logging the use of an applicator.

### SUMMARY

It is an object of the disclosure to provide an improved brachytherapy system.

The aforementioned object can, for example, be achieved by a brachytherapy system having: a radiation generation apparatus configured to generate x-ray radiation; a controller for controlling the radiation generation apparatus; an applicator embodied in such a manner that it can be placed on the radiation generation apparatus; an information transmission system configured to capture an information item embodied in or at the applicator if the applicator has been placed on the radiation generation apparatus and further configured to transmit the information item to the controller; and, the controller being configured to control the generation of x-ray radiation by the radiation generation apparatus on a basis of the information item captured by the information transmission system.

### BRIEF DESCRIPTION OF THE DRAWINGS

The invention will now be described with reference to the drawings wherein:

FIG. 1 shows a schematic illustration of a brachytherapy system;

FIG. 2 shows a schematic illustration of an information transmission system in accordance with one embodiment;

FIG. 3 shows a schematic illustration of an applicator in the plane A-A;

FIG. 4 shows a schematic illustration of an information transmission system in accordance with a further embodiment;

FIG. 5 shows a schematic illustration of an information transmission system in accordance with a further embodiment;

FIG. 6 shows a schematic illustration of an information transmission system in accordance with a further embodiment;

FIG. 7 shows a method for applying the brachytherapy system; and,

FIG. 8 shows a further method for applying the brachytherapy system.

### DESCRIPTION OF THE PREFERRED EMBODIMENTS

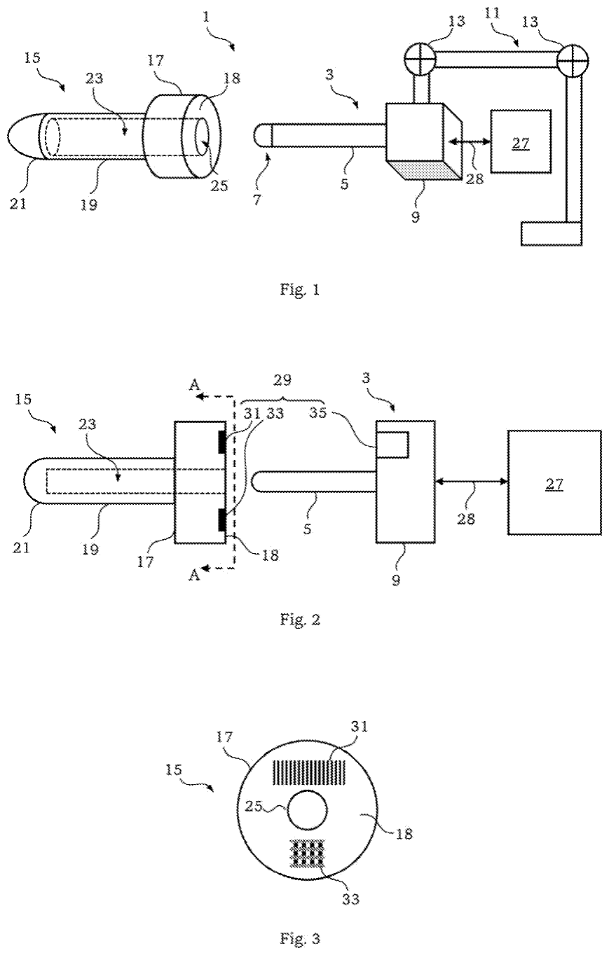

FIG. 1 shows a schematic illustration of a brachytherapy system 1. The brachytherapy system 1 includes a radiation generation apparatus 3, which is configured to generate x-ray radiation. The radiation generation apparatus 3 includes a tube 5, at the end of which x-ray material 7 is arranged. At the other end of the tube 5, the radiation generation apparatus 3 includes a housing 9, contained in which there is a particle beam apparatus, not illustrated in the figures, which is configured to generate a particle beam and direct the latter through the tube 5 at the x-ray material 7. X-ray radiation, which can be used for therapeutic treatment, is generated by the interaction of the particle beam with the x-ray material 7.

The radiation generation apparatus 3 is held by a movable stand 11. In the example shown in FIG. 1, the stand 11 includes a plurality of joints 13, which allow variable positioning and orientation of the radiation generation apparatus 3. The stand 11 can be automated. By way of example, the stand 11 includes one or more actuators, which are able to adjust the joints 13. The stand 11 can be controlled by a controller 27 of the brachytherapy system 1.

The brachytherapy system 1 further includes an applicator 15, which is embodied in such a way that it can be placed on the radiation generation apparatus 3. For the therapeutic treatment of tissue, the applicator 15 is introduced into the tissue. Depending on the desired irradiation, the applicator 15 can have different shapes and dimensions. In practice, a multiplicity of applicators with different sizes and configurations are provided for the operator of the brachytherapy system 1, with each applicator being able to be placed on the radiation generation apparatus 3.

The applicator 15 includes a connection section 17, a tubular section 19 and a tip 21.

The connection section 17 and the tubular section 19 have a contiguous cavity 23, which opens into an opening 25 in the connection section 17. The opening 25 and the cavity 23 are embodied in such a way that the tube 5 of the radiation generation apparatus 3 can be inserted into the cavity 23 of the applicator 15 through the opening 25. The cavity 23 can extend into the tip 21 of the applicator 15. The applicator 15 is placed on the radiation generation apparatus 3 by virtue of the tube 5 of the radiation generation apparatus 3 being guided into the cavity 23 of the applicator 15 through the opening 25 of the applicator 15.

The tip 21 can be rounded off, as shown in FIG. 1. Alternatively, the tip can have a different form, for example a spherical form or the like.

A retaining apparatus, not illustrated in the figures, can be arranged at the housing 9 of the radiation generation apparatus 3 and the connection section 17 of the applicator 15, the retaining apparatus being configured to provide a releasable connection between the housing 9 of the radiation generation apparatus 3 and the connection section 17 of the applicator 15. As a result thereof, the applicator 15 can be held at the radiation generation apparatus 3. Moreover, the applicator 15 can be detached and removed from the radiation generation apparatus 3 without suffering damage. Examples of such a retaining apparatus are a bayonet closure, a magnetic closure or the like.

The brachytherapy system 1 further includes a controller 27, which is configured to control the radiation generation apparatus 3. By way of example, the controller 27 controls the generation of x-ray radiation by the radiation generation apparatus 3. The controller 27 can further control the stand 11. Further functions of the controller 27 are explained below with reference to the further figures.

The brachytherapy system 1 further includes an information transmission system. An information transmission system 29 and the use thereof is explained with reference to FIGS. 2 and 3.

FIG. 2 shows a schematic illustration of the applicator 15 and the radiation generation apparatus 3 from the side. FIG. 3 shows a surface 18 of the connection section 17 of the applicator 15, which faces the housing 9 of the radiation generation apparatus 3 if the applicator 15 has been placed on the radiation generation apparatus 3. The perspective of FIG. 3 is represented by the section label A-A in FIG. 2.

The information transmission system 29 is configured to capture an information item embodied in or at the applicator 15 if the applicator 15 has been placed on the radiation generation apparatus 3. Further, the information transmission system 29 is configured to transmit the captured information item to the controller 27 if the applicator 15 has been placed on the radiation generation apparatus 3.

The information transmission system 29 can be configured in such a way that the information item cannot be captured if the applicator 15 has not been placed on the radiation generation apparatus 3. Accordingly, the information item can only be captured if the applicator 15 has been placed on the radiation generation apparatus 3. Moreover, the information transmission system 29 can be configured in such a way that the captured information item cannot be transmitted to the controller 27 if the applicator 15 has not been placed on the radiation generation apparatus 3. Accordingly, the captured information item can only be transmitted to the controller 27 if the applicator 15 has been placed on the radiation generation apparatus 3.

In the example of FIGS. 2 and 3, the information transmission system 29 includes an optical label 31, 33 and a reader 35. By way of example, the optical label can be a barcode 31, a QR code 33 or the like. By way of example, the optical label 31, 33 is arranged at the surface 18 of the applicator 15, which faces the housing 9 of the radiation generation apparatus 3 if the applicator 15 has been placed on the radiation generation apparatus 3. The reader 35 is configured to read the optical label 31, 33. The information item embodied by the optical label 31, 33 at the applicator 15 can consequently be read by the reader 35. The reader 35 can transmit the information item captured by reading the optical label 31, 33 to the controller 27. A communications link 28 between the reader 35 or the radiation generation apparatus 3 and the controller 27 for transmitting the information item is represented schematically by a double-headed arrow.

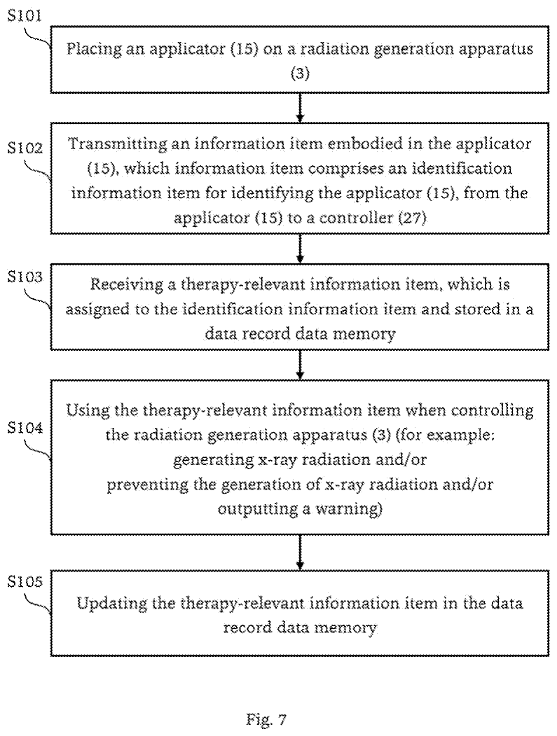

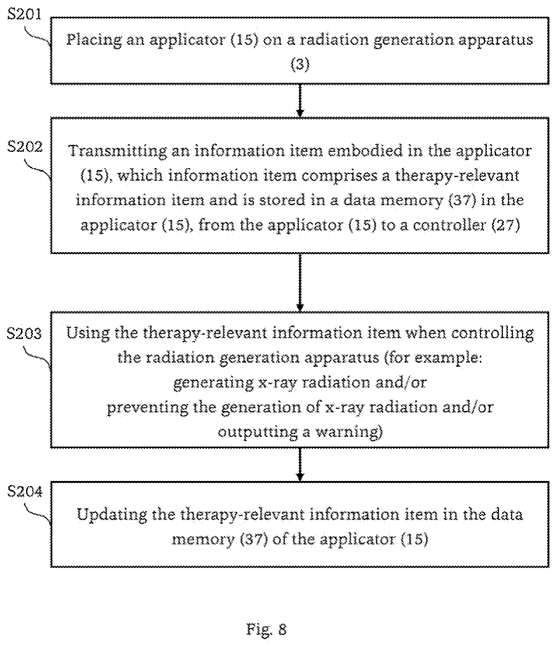

The controller 27 receives the information item that was captured and transmitted by the information transmission system 29 and uses this information item in conjunction with the generation of x-ray radiation by the radiation generation apparatus 3. Expressed differently, the controller 27 controls the generation of x-ray radiation by the radiation generation apparatus 3 on the basis of the captured and received information item. Further details about the significance of the information item and the use thereof for generating the x-ray radiation are explained below with reference to FIGS. 7 and 8.

In the example shown in FIG. 2, the reader 35 is integrated in the radiation generation apparatus 3. However, the reader 35 can also be provided separately from the radiation generation apparatus 3. However, it is essential that the information item in the form of the optical label 31, 33 embodied in or at the applicator 15 can (only) be captured if the applicator 15 has been placed on the radiation generation apparatus 3.

The fact that the information item embodied in or at the applicator 15 can (only) be captured if the applicator 15 has been placed on the radiation generation apparatus 3 ensures that the information item which is provided for the controller by the information transmission system 29 actually originates from the applicator 15 that has been placed on the radiation generation apparatus 3. Consequently, the controller 27 can use the information item originating from the applied applicator 15 for the purposes of controlling the radiation generation apparatus 3. Accordingly, a risk of confusion in respect of the applicator 15 used for the irradiation is eliminated since the information item need not be captured by, for instance, a person but instead is captured at the applicator 15 by the information transmission system 29 and transmitted to the controller 27.

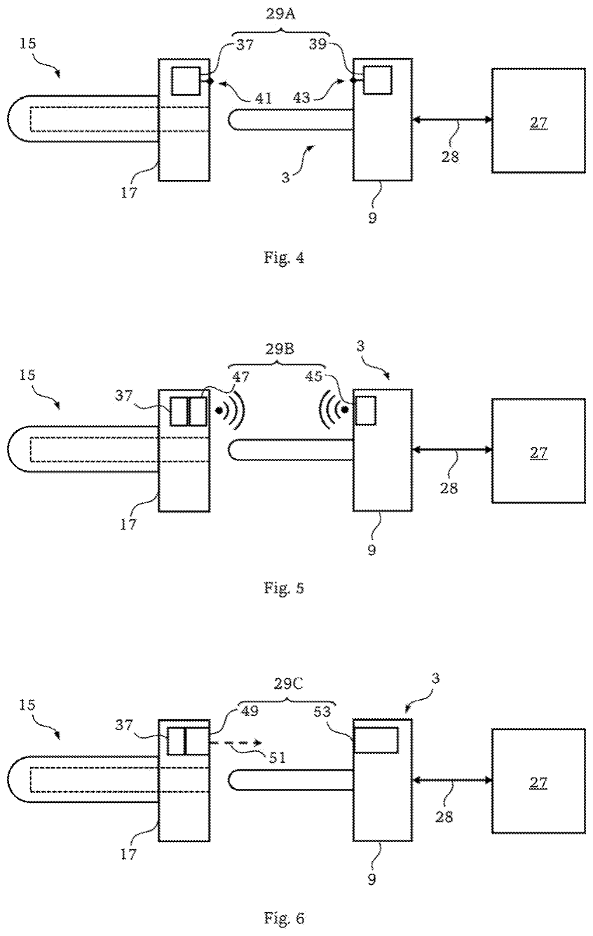

Alternative information transmission systems are explained with reference to FIGS. 4 to 6.

FIG. 4 shows a schematic illustration of an information transmission system 29A in accordance with a further embodiment. In FIG. 4, the applicator 15 and the radiation generation apparatus 3 are shown from the side. The information transmission system 29A includes a data memory 37, which is arranged in the applicator 15. In this embodiment, the information item embodied in the applicator 15 is realized by a signal stored in the data memory 37.

The information transmission system 29A further includes a receiver unit 39. The receiver unit 39 is configured to receive the information item from the data memory 37 if the applicator 15 has been placed on the radiation generation apparatus 3. As shown in FIG. 4, the receiver unit 39 can be integrated in the radiation generation apparatus 3. However, the receiver unit 39 can also be arranged outside of the radiation generation apparatus 3 provided the receiver unit 39 can receive the information item realized in the data memory 37 (only) if the applicator 15 has been placed on the radiation generation apparatus 3. If the receiver unit 39 has received the information item, the receiver unit 39 transmits the information item to the controller 27.

In the example shown in FIG. 4 the applicator 15 has at its surface at least one electrical contact 41, which provides a communications link to the data memory 37. The electrical contact 41 is situated on the surface 18 of the connection section 17 of the applicator 15, which faces the housing 9 of the radiation generation apparatus 3 if the applicator 15 has been placed on the radiation generation apparatus 3.

At its surface, the housing 9 of the radiation generation apparatus 3 includes at least one electrical contact 43, which fits to the at least one electrical contact 41 of the applicator 15. The at least one electrical contact 43 provides a communications link to the receiver unit 39.

If the applicator 15 has been placed on the radiation generation apparatus 3, the at least one electrical contact 41 and the at least one electrical contact 43 are electrically interconnected, as a result of which a wired communications link is established between the data memory 37 and the receiver unit 39. Consequently, the receiver unit 39 can receive the information item from the data memory 37 if the applicator 15 has been placed on the radiation generation apparatus 3. The communications link is interrupted if the applicator 15 is not placed on the radiation generation apparatus 3 and the receiver unit 39 cannot receive the information item. Consequently, the receiver unit 39 can only read the information item realized by the signal in the data memory 37 if the applicator 15 has been placed on the radiation generation apparatus 3.

The receiver unit 39 transmits the information item received to the controller 27. Consequently, the controller 27 can use the information item received for controlling the radiation generation apparatus 3.

FIG. 5 shows a schematic illustration of an information transmission system 29B in accordance with a further embodiment. The information transmission system 29B is configured to wirelessly transmit to a receiver unit 45 the information item realized in the data memory 37 in the form of the signal. To this end, the information transmission system 29B includes a communications module 47 arranged in or at the applicator 15. The communications module 47 is configured to read the information item from the data memory 37 and wirelessly transmit the latter to the receiver unit 45. Accordingly, the communications module 47 is configured to wirelessly emit the information item; and the receiver unit 45 is configured to receive the information item transmitted by the communications module 47.

In the example shown in FIG. 5, the receiver unit 45 is integrated in the radiation generation apparatus 3. However, the receiver unit 45 can also be arranged outside of the radiation generation apparatus 3.

The communications module 47 and the receiver unit 45 can be configured in such a way that the wireless communication only works over a short distance. This ensures that the transmission of the information item from the applicator 15 to the receiver unit 45 can only be implemented if the applicator 15 has been placed on the radiation generation apparatus 3.

If the receiver unit 45 has received the information item, the receiver unit 45 transmits the information item to the controller 27. The controller 27 uses the received information item for controlling the radiation generation apparatus 3.

By way of example, the communications module 47 can be an RFID (radio frequency identification) tag. The RFID tag can be arranged at the surface of the applicator 15 or in the interior of the applicator 15.

FIG. 6 shows a schematic illustration of an information transmission system 29C in accordance with a further embodiment. In the example shown in FIG. 6, the information transmission system 29C includes an optical information transmission system. The optical information transmission system is configured to use a light signal 51 for transmitting the information item stored in the data memory 37. By way of example, the information transmission system 29C includes a controllable light source 49, which is arranged in the applicator 15 and connected to the data memory 37. The controllable light source 49 is configured to emit a light signal 51, which carries the information item realized in the data memory 37. The information transmission system 29C further includes a receiver unit 53, which is configured to (only) receive the light signal 51 if the applicator 15 has been placed on the radiation generation apparatus 3. In the example shown in FIG. 6, the receiver unit 53 is integrated in the radiation generation apparatus 3. However, the receiver unit 53 can also be arranged outside of the radiation generation apparatus 3 provided the receiver unit 53 can receive the light signal 51 if the applicator 15 has been placed on the radiation generation apparatus 3.

The receiver unit 53 transmits the information item received to the controller 27. The controller 27 uses the received information item for controlling the radiation generation apparatus 3.

Various information transmission systems, which are configured to capture the information item embodied in or at the applicator 15 and transmit the information item to the controller 27 if the applicator 15 has been placed on the radiation generation apparatus 3, were described with reference to FIGS. 2 to 6. Below, different embodiments in respect of the meaning of the information item and in respect of the use of the information item by the controller 27 are explained with reference to FIGS. 7 and 8.

FIG. 7 shows a method for using the brachytherapy system 1 explained in conjunction with FIGS. 1 to 6.

An applicator 15 is placed on a radiation generation apparatus 3 in a step S101. This is carried out by virtue of the tube 5 of the radiation generation apparatus 3 being inserted through the opening 25 in the connection section 17 of the applicator 15 and, further, being inserted in the cavity 23 of the applicator 15.

In step S102, which follows step S101, an information item embodied in or at the applicator 15 is transmitted from the applicator 15 to the controller 27 by way of the information transmission system 29, 29A, 29B, 29C, wherein the information item is an identification information item, which (uniquely) identifies the applicator 15. The identification information item serves for the (unique) identification of the applicator 15. By way of example, the identification information item can be a number which is realized in the form of an optical label 31, 33 or as a signal in the data memory 37.

A data record is assigned to the identification information item. The data record includes a therapy-relevant information item. Hence, a therapy-relevant information item is assigned to an identification information item.

In step S103, which follows step S102, the controller 27 receives from a data record data memory the data record assigned to the identification information item. By way of example, the data record data memory is a different memory to the data memory 37. By way of example, the data record data memory can be implemented in the form of a hard disk drive, a general storage medium, in a distributed network and the like. A data record containing a therapy-relevant information item about the respective applicator can be stored in the data record data memory for each of a multiplicity of applicators. The identification information item allows unique mapping between an applicator and a data record, with the data record only relating to a single applicator.

The therapy-relevant information item includes an information item which characterizes the applicator and is relevant to an irradiation.

By way of example, the therapy-relevant information item includes a type designation of the applicator 15. Applicators can have different shapes. The various shapes can each have assigned a type designation, which specifies the shape of the respective applicator. Examples of the type designation may include: “needle applicator”, “spherical head applicator” and the like.

By way of example, the therapy-relevant information item includes a size of the applicator 15. The size of the applicator is an essential criterion when selecting an applicator in brachytherapy since the applicator, for example, should fill as ideally as possible a cavity in the tissue to be irradiated and should support the tissue.

By way of example, the therapy-relevant information item includes a batch number of the applicator 15 and/or a serial number of the applicator 15. By way of example, the batch number specifies a production cycle during the production process of applicators. By way of example, the serial number is a designation for the production criteria of the applicator.

By way of example, the therapy-relevant information item includes a use state of the applicator 15. By way of example, the use state can be “new” or “used” or “used up”. The use state is “new” if the applicator has not yet been used for an irradiation. The use state can be altered to “used” or “used up” by the controller once the applicator has been used for an irradiation.

By way of example, the therapy-relevant information item includes a use duration of the applicator 15. By way of example, the use duration specifies the time duration or the frequency with which the applicator has been used for an irradiation.

By way of example, the therapy-relevant information item includes a radiation dose of the applicator 15. The radiation dose of the applicator 15 specifies the overall radiation dose that has acted on the applicator 15 over previous uses.

By way of example, the therapy-relevant information item includes an expiry date of the applicator 15. Applicators are provided in a sterile state in a packaging. However, the packaging only ensures the sterile state up to the expiry date. For this and other reasons, an applicator must only be used up to the expiry date assigned thereto.

The therapy-relevant information item can contain some or all of the aforementioned information items and further information items that have not been specified.

In step S104, which follows step S103, the controller 27 uses the received therapy-relevant information item when controlling the radiation generation apparatus 3. Expressed differently, the controller 27 controls the generation of x-ray radiation by the radiation generation apparatus 3 on the basis of the received therapy-relevant information item. Examples for the use of the therapy-relevant information item by the controller 27 are explained below.

In an optional step S105, which follows step S104, there is updating of the therapy-relevant information item in the data record data memory. By way of example, the controller 27 changes the therapy-relevant information item in the data record in the data record data memory on the basis of an irradiation carried out. In this way, the therapy-relevant information item relating to the applicator used for carrying out the irradiation is updated, and so the therapy-relevant information item relating to this applicator is up-to-date.

A method in which the information item embodied in or at the applicator 15 includes an identification information item was described with reference to FIG. 7. The identification information item serves for the (unique) identification of the applicator intended to be used for an irradiation. The therapy-relevant information item in respect of this applicator is obtained on the basis of the identification information item. In this way, it is sufficient for the information item embodied in or at the applicator to merely include an identification information item. The information item embodied in or at the applicator need not include the therapy-relevant information item itself. Accordingly, simple identification apparatuses can be used to carry out this method, for example the optical labels 31, 33 (see FIG. 3). The data memory 37 need only store a single information item, specifically the identification information item itself. Therefore, the data memory 37 can have a simple and cost-effective configuration. The data record which contains the therapy-relevant information item in relation to an applicator can be stored outside of the applicator 15 in the data record data memory. By way of example, the data record data memory can be a server or the like, which provides a central access point for the therapy-relevant information of a multiplicity of applicators.

Now, a method for using the brachytherapy system 1, in which the information item embodied in or at the applicator 15 includes the therapy-relevant information item itself, is explained with reference to FIG. 8. By way of example, this method can be carried out with the brachytherapy system 1 described with reference to FIGS. 4 to 6, in which the therapy-relevant information item is contained in the data memory 37 which is arranged in the applicator 15.

The applicator 15 is placed on the radiation generation apparatus 3 in step S201. This step corresponds to step S101 of the method shown in FIG. 7.

In step S202, which follows step S201, the information transmission system 29A, 29B, 29C transmits to the controller 27 the information item embodied in the applicator 15, wherein the information item includes the therapy-relevant information item. The therapy-relevant information item is stored in the data memory 37 in the applicator 15. In contrast to the method of FIG. 7, in which the therapy-relevant information item was received by the controller 27 from a data record data memory outside of the applicator 15 using an identification information item, the therapy-relevant information item is stored in the applicator 15 or in the data memory 37 in the applicator 15 and transmitted from there to the controller 27 using the information transmission system 29A, 29B, 29C in the method in accordance with FIG. 8.

In step S203, which follows step S202, the controller 27 uses the received therapy-relevant information item for controlling the radiation generation apparatus 3. Step S203 corresponds to step S103 of FIG. 7.

In an optional step S204, which follows step S203, there is updating of the therapy-relevant information item in the data memory 37 of the applicator 15. By way of example, the controller 27 instructs the information transmission system 29 to alter the therapy-relevant information item in the data memory 27 on the basis of an irradiation performed.

By way of example, the controller 27 adds to the therapy-relevant information item in the data memory 27 an information item relating to the use of the applicator (15) during the irradiation and/or an irradiation duration of the applicator (15) during the irradiation and/or a dose applied to the applicator (15) and/or updates a corresponding information item in the therapy-relevant information item in the data memory 27.

Some examples for using the therapy-relevant information item when controlling the radiation generation apparatus 3 as per steps S104 and S204 are explained below.

By way of example, the controller compares the therapy-relevant information item with a radiation treatment plan which defines the irradiation to be performed. On the basis of the result of the comparison, the controller controls the generation of x-ray radiation by the radiation generation apparatus 3 and outputs warnings where necessary.

A radiation treatment plan can define a specified type designation. The controller 27 can compare the specified type designation specified in the radiation treatment plan with the type designation in the therapy-relevant information item and can prevent the generation of x-ray radiation if the comparison yields that the type designation of the applicator 15 that has been placed on the radiation generation apparatus 3 does not correspond to the specified type designation.

A radiation treatment plan can define a specified size of the applicator to be used. The controller 27 can compare the specified size specified in the radiation treatment plan with the size of the applicator in the therapy-relevant information item and can prevent the generation of x-ray radiation if the comparison yields that the size of the applicator 15 that has been placed on the radiation generation apparatus 3 does not correspond to the specified size.

A radiation treatment plan can define a specified batch number and/or a specified serial number of the applicator to be used. The controller 27 can compare the specified batch number/serial number specified in the radiation treatment plan with the batch number/serial number of the applicator in the therapy-relevant information item and can prevent the generation of x-ray radiation if the comparison yields that the batch number/serial number of the applicator 15 that has been placed on the radiation generation apparatus 3 does not correspond to the specified batch number/serial number.

A radiation treatment plan can define a specified use state. The controller 27 can compare the specified use state specified in the radiation treatment plan with the use state in the therapy-relevant information item and can prevent the generation of x-ray radiation if the comparison yields that the use state of the applicator 15 that has been placed on the radiation generation apparatus 3 does not correspond to the specified use state.

A radiation treatment plan can define an admissible use duration. The controller 27 can compare the admissible use duration specified in the radiation treatment plan with the use duration in the therapy-relevant information item and can prevent the generation of x-ray radiation if the comparison yields that the use duration of the applicator 15 that has been placed on the radiation generation apparatus 3 exceeds the admissible use duration.

A radiation treatment plan can define an admissible radiation dose. The controller 27 can compare the admissible radiation dose specified in the radiation treatment plan with the radiation dose in the therapy-relevant information item and can prevent the generation of x-ray radiation if the comparison yields that the radiation dose of the applicator 15 that has been placed on the radiation generation apparatus 3 exceeds the admissible radiation dose.

By way of example, the controller 27 can be configured to prevent the generation of x-ray radiation if the controller determines that an expiry date of the applicator specified in the therapy-relevant information item has passed. In this embodiment, the controller checks the expiry date and prevents the generation of x-ray radiation if the expiry date has passed. This ensures that irradiation can only be performed with a sufficiently safe applicator.

In addition or as an alternative to the prevention of the generation of x-ray radiation, the controller can output warnings on the basis of the therapy-relevant information item, which warnings draw the attention of a user of the brachytherapy system to the fact that the applicator currently placed on the radiation generation apparatus 3 does not correspond to the radiation treatment plan.

The brachytherapy system 1 described above increases the safety when carrying out therapeutic irradiation processes. This can ensure that the applicator used corresponds to a radiation treatment plan. Errors as a result of human failure can be precluded in this case since either the applied applicator can be identified unambiguously by an identification information item or the therapy-relevant information item relating to the applicator is stored in the applicator itself and transmitted to the controller if the applicator has been placed on the radiation generation apparatus.

It is understood that the foregoing description is that of the preferred embodiments of the invention and that various changes and modifications may be made thereto without departing from the spirit and scope of the invention as defined in the appended claims.

What is claimed is:

1. A brachytherapy system comprising:
a radiation generation apparatus configured to generate x-ray radiation;
a controller for controlling said radiation generation apparatus;
an applicator embodied in such a manner that it can be placed on said radiation generation apparatus;
an information transmission system configured to capture an information item embodied in or at said applicator if said applicator has been placed on said radiation generation apparatus and further configured to transmit the information item to said controller; and,
said controller being configured to control the generation of x-ray radiation by the radiation generation apparatus on a basis of the information item captured by said information transmission system.

2. The brachytherapy system of claim 1, wherein the information item is realized by an optical label and said information transmission system includes a reader configured to read the optical label at said applicator if said applicator has been placed on said radiation generation apparatus.

3. The brachytherapy system of claim 1 further comprising:
a data memory arranged in the applicator;
the information item being realized by a signal in said data memory; and,
said information transmission system including a receiver unit configured to receive the information item from said data memory if said applicator has been placed on said radiation generation apparatus.

4. The brachytherapy of claim 3, wherein said applicator defines a surface and has at said surface at least one electrical contact for establishing a wired communications link between said data memory and said receiver unit.

5. The brachytherapy system of claim 3, wherein said information transmission system includes a communications module arranged in or at said applicator;
and, said communications module is configured to wirelessly transmit the information item to said receiver unit.

6. The brachytherapy system of claim 3, wherein said information transmission system includes an optical information transmission system configured to output a light signal for transmitting the information item.

7. The brachytherapy system of claim 1, wherein
the information item includes an identification information item which identifies said applicator;
said controller is further configured to receive, from a data record data memory, a data record assigned to the identification information item, wherein the data record includes a therapy-relevant information item; and
said controller is configured to control the generation of x-ray radiation by said radiation generation apparatus on a basis of the received therapy-relevant information item.

8. The brachytherapy system of claim 7, wherein said controller is further configured to alter the therapy-relevant information item in the data record in the data record data memory on a basis of an irradiation performed.

9. The brachytherapy system of claim 3, wherein the information item includes a therapy-relevant information item; and, said controller is configured to control the generation of x-ray radiation by the radiation generation apparatus on a basis of the received therapy-relevant information item.

10. The brachytherapy system of claim 9, wherein said controller is further configured to alter the therapy-relevant information item in said data memory in said applicator on a basis of an irradiation performed.

11. The brachytherapy system of claim 8, wherein said controller is further configured to add to the therapy-relevant information item an information item relating to at least one of a use of said applicator during the irradiation, an irradiation duration of said applicator during the irradiation, and a dose applied to said applicator during the irradiation.

12. The brachytherapy system of claim 8, wherein said controller is further configured to update in the therapy-relevant information item an information item relating to at least one of a use of said applicator during the irradiation, an irradiation duration of said applicator during the irradiation, and a dose applied to said applicator during the irradiation.

13. The brachytherapy system of claim 7, wherein the therapy-relevant information item includes at least one of: a type designation of said applicator, a size of said applicator, a batch number of said applicator, a serial number of said applicator, a use state of said applicator, a use duration of said applicator, a radiation dose of said applicator, and an expiry date of said applicator.

14. The brachytherapy system of claim 7, wherein said controller is configured to compare the therapy-relevant information item with a radiation treatment plan and, on a basis of a result of the comparison, to control the generation of x-ray radiation and an outputting of warnings.

15. The brachytherapy system of claim 3, wherein said data memory is a non-transitory computer readable storage medium.
